(19) United States
(12) Patent Application Publication
(10) Pub. No.: US 2015/0118649 A1
(43) Pub. Date: Apr. 30, 2015

(54) SURFACE TREATMENT METHOD FOR IMPLANT

(71) Applicant: National Taiwan University, Taipei (TW)

(72) Inventors: Sheng-Hao HSU, Taipei (TW); Wan-Yu TSENG, Taipei (TW); Li-Deh LIN, Taipei (TW); Ming-Shu LEE, Taipei (TW); Ming-Hung TSENG, Taipei (TW); Wei-Fang SU, Taipei (TW); Feng-Yu TSAI, Taipei (TW); Min-Huey CHEN, Taipei (US)

(73) Assignee: National Taiwan University, Taipei (TW)

(21) Appl. No.: 14/282,122

(22) Filed: May 20, 2014

(30) Foreign Application Priority Data
Oct. 25, 2013 (TW) 102138619

(51) Int. Cl. A61C 8/00; C23C 16/40; C23C 16/455; A61F 2/28; A61F 2/82

(52) U.S. Cl. CPC A61C 8/0013; A61F 2/28; A61F 2/82; C23C 16/45525; C23C 16/403; C23C 16/407; C23C 16/405
USPC 433/201.1; 623/23.53; 623/1.15; 427/2.24; 427/2.25; 427/2.27

(57) ABSTRACT

The present invention relates to a surface treatment method for an implant, comprising: providing an implant; and forming a ceramic layer on a surface of the implant by atomic layer deposition, wherein the ceramic layer has a thickness of 5-150 nm; a root mean square roughness increase in a range of 15 nm or less; and a friction coefficient of 0.1-0.5. The ceramic layer formed on the surface of the implant can fully encapsulate the surface of the implant with excellent uniformity to effectively block the free metal ions dissociated from the implant. Moreover, it has anti-oxidation and anti-corrosion effects, and greatly enhances the biocompatibility of the implant.

### CROSS REFERENCE TO RELATED APPLICATION

This application claims the benefits of the Taiwan Patent Application Serial Number 102138619, filed on Oct. 25, 2013, the subject matter of which is incorporated herein by reference.

### BACKGROUND OF THE INVENTION

1. Field of the Invention

The present invention relates to a surface treatment method for an implant, and more particularly to a surface treatment method for a dental implant, an orthopedic implant or a cardiovascular stent.

2. Description of Related Art

An implant is a medical device for replacement or support of a damaged site or function to treat the disease or restore the normal function of the damaged site in vivo. Dental implants, an orthopedic implant, a cardiovascular stent or so on are implanted in the patient permanently or semi-permanently, and thus the selection of the implant material is very important. When an implant is implanted in vivo, not only immune reaction or rejection reaction will be induced, but also biotoxicity which causes adverse reactions of the surrounding tissues will occur. Therefore, so far, surface treatment methods are employed to produce a rough surface on the implant surface or form a metal oxide layer, in order to enhance biocompatibility.

Dental implants used in dentistry are required to have aesthetic appearances, and typical materials of the dental implants are, for example, commercial pure titanium (CPT), or Ti₆Al₄V etc., which mostly exhibit a metallic color of gray to dark gray. When the implants are exposed or the gum is too thin, the surface color of the implants will cause aesthetic problems.

Currently, a typical surface treatment for the implant comprises acid etching, sandblasting or formation of a biomedical ceramic layer on the surface of the implant. The biomedical ceramic layer is typically a metal oxide layer, such as Al₂O₃, TiO₂, ZrO₂ etc. CN 102345134A discloses a surface treatment method for a dental implant, comprising treating a titanium or titanium alloy surface by sandblasting, followed by immersion etching in an acid solution; and then forming an oxide layer on the surface using plasma enhanced chemical vapor deposition (PECVD). However, the acid etching is likely to cause excessive corrosion, and it is difficult to control the surface structure of the implant. Moreover, the oxide layer formed by plasma enhanced chemical vapor deposition may easily crack, or have poor adhesion. On the other hand, TWI385004 discloses a surface treating method for titanium artificial implant, comprising: connecting a titanium artificial implant and a cathode and placing the implant in an electrolyte with power supply to form a titanium oxide layer on the surface of the titanium artificial implant. However, the formed titanium dioxide layer may greatly change the surface morphology, and a high-precision thickness control is difficult and the cracking and poor adhesion problems still exist. In addition, if the implant is made of non-titanium or non-aluminum alloy materials such as stainless steel, a biomedical ceramic layer cannot be formed on the surface by anodic oxidation. Furthermore, if the surface roughness of the biomedical ceramic layer on the surface of the implant is too high, it will cause excessive friction between the biomedical ceramic layer and the tissues during implanting. In addition, detachment or damage due to the cracking and poor adhesion during implanting will induce foreign body reactions of the surrounding tissues.

Therefore, what is urgently needed is to provide a surface treatment method for an implant, to form a ceramic layer with a better biocompatibility, an excellent coating property and good adhesion on the implant surface, and to change the surface color of the implant to meet the aesthetic requirements.

### SUMMARY OF THE INVENTION

An object of the present invention is to provide a surface treatment method for an implant, comprising: providing an implant; and forming a ceramic layer on a surface of the implant by atomic layer deposition, wherein the ceramic layer has a thickness of 5-150 nm; a root mean square roughness increase in a range of 15 nm or less after deposition. Further, in an aspect of the present invention, the ceramic layer has a friction coefficient of 0.1-0.5. In this case, the ceramic layer preferably has a thickness ranging from 20 to 120 nm, a root mean square roughness increase in a range of 10 nm or less. Also, in an aspect of the present invention, the ceramic layer preferably has a friction coefficient ranging from 0.1 to 0.35. In order to achieve the desired thickness of the ceramic layer, the ceramic layer is formed by repeating the atomic layer deposition for 10-5000 times. The ceramic layer is made of a metal oxide selected from the group consisting of Al₂O₃, ZnO, TiO₂, ZrO₂, HfO₂, and a mixture thereof, preferably TiO₂, HfO₂ and ZrO₂, and more preferably ZrO₂. In addition, the atomic layer deposition for forming the ceramic layer is performed at a reaction temperature of 25-450° C., and preferably 150-250° C. Such a temperature range allows the formed ceramic layer of TiO₂, HfO₂, or ZrO₂ to be in a crystalline structure, and does not result in recrystallization of most of metal substrates, or cracking of polymer substrates. When the ceramic layer deposited on the surface of the implant is a crystalline structure, the ceramic layer is difficult to dissolve into the body fluid and enter the body, and therefore a more stable ceramic layer on the surface of the implant can be provided with a reduced toxicity. In the surface treatment method for an implant provided by the present invention, the implant is preferably a dental implant, an orthopedic implant, or a cardiovascular stent.

In the surface-treated implant provided by the present invention, the ceramic layer is formed on the surface of the implant by atomic layer deposition. Since the atomic layer deposition is based on surface molecular monolayer adsorption, it has a self-limiting nature, that is, only a thickness of a molecular monolayer is deposited in one deposition cycle. Therefore, the thickness of the ceramic layer may be controlled by setting the number of the deposition cycles, and the ceramic layer deposited by the atomic layer deposition has excellent continuity and uniformity, and is able to overcome the shielding effect caused by the steric structure or surface morphology of the implant. Therefore, the ceramic layer can be evenly coated on the entire surface of the implant to effectively block the free metal ions dissociated from the implant and provide anti-oxidation and anti-corrosion effects. Moreover, the ceramic layer is not prone to cracking, poor adhesion, and other problems. In addition, the ceramic layer may be formed to be crystalline on the substrate surface by controlling the temperature of the atomic layer deposition process. The crystalline ceramic layer is difficult to dissolve in water, and thus, difficult to dissolve into the body fluid and impact the body, which is particularly desirable for the implant which needs a long-term contact with the body fluid.

Furthermore, the implant with the ceramic layer deposited thereon provided by the present invention has a significantly enhanced biocompatibility. In an aspect of the present invention, the implant with the ceramic layer of ZrO₂, HfO₂, or TiO₂ deposited thereon has a significantly decreased bio-toxicity with the increase in the thickness of the ceramic layer, indicating that the bio-toxicity of the implant is successfully reduced and the biocompatibility of the implant is improved. In addition, in an aspect of the present invention, it can be clearly observed that the thicker the ceramic layer deposited on the surface, the higher the surface friction and roughness.

Furthermore, in the present invention, the color of the implants varies with the material of the deposited ceramic layer, and may vary with the thickness thereof. For example, when ZrO₂ is deposited on the surface of a substrate of titanium alloy (Ti₆Al₄V) using the atomic layer deposition, with the increase in thickness of the ceramic layer, the color of the substrate can gradually transform from gray to yellow. Therefore, the implants with various colors may be obtained by using different materials of the ceramic layer and controlling the thickness of the deposited layer, to improve the aesthetic appearance of the implant that is easily exposed (e.g. a dental implant).

Accordingly, the present invention provides a surface treatment method for an implant, wherein a ceramic layer is formed on a surface of the implant by atomic layer deposition, and the formed ceramic layer can fully encapsulate the surface of the implant with excellent uniformity, and the ceramic layer is quite stable, not easily peeled off, and able to effectively block the free metal cations dissociated from the implant. Moreover, it provides anti-oxidation and anti-corrosion effects, and greatly enhances the biocompatibility of the implant. For implants needed to be exposed, it can provide a color changing, and aesthetic effect.

### BRIEF DESCRIPTION OF THE DRAWINGS

FIG. 1 shows a schematic diagram of the result of the lactate dehydrogenase analysis according to Test Example 1 of the present invention.

FIG. 2 shows a schematic diagram of the result of the X-ray photoelectron spectroscopy analysis according to Test Example 2 of the present invention.

FIG. 3 shows a schematic diagram of the result of the X-ray diffraction analysis of Example 7 according to Test Example 3 of the present invention.

FIG. 4 shows a schematic diagram of the result of the X-ray diffraction analysis of Example 13 according to Test Example 3 of the present invention.

FIG. 5 shows a schematic diagram of the friction according to Test Example 3 of the present invention.

FIG. 6 shows a schematic diagram of the roughness according to Test Example 4 of the present invention.

### DETAILED DESCRIPTION OF THE PREFERRED EMBODIMENT

### Example 1

In this Example, a pure titanium (Ti) cylinder of 14 mm diameter and 2 mm height was provided as the substrate. Then, the atomic layer deposition was performed in an atomic layer deposition reactor (Savannah S100, manufactured by CambrigeNanoTech Ltd.) with tetrakis dimethylamino zirconium (TDMAZ; Zr(N(CH₃)₂)₄) and water as the precursors at 150° C., to form a ZrO₂ layer on the pure titanium substrate. The atomic layer deposition method is performed by the following steps: (1) application of pulse of zirconium dimethyl ammonium; (2) nitrogen purging; (3) application of pulse of water; and (4) nitrogen purging, which were repeated for more than 200 times, to provide ZrO₂ with a thickness of 20 nm. Thereby, a ZrO₂ ceramic layer having a thickness of 20 nm was formed on the pure titanium substrate.

### Example 2

In Example 2 the same method as in Example 1 was performed to form the ZrO₂ layer on the pure titanium substrate, except that the atomic layer deposition cycle was repeated for more than 1000 times to provide ZrO₂ with a thickness of 100 nm. Thereby, a ZrO₂ ceramic layer having a thickness of 100 nm was formed on the pure titanium substrate.

### Example 3

In this Example, a titanium alloy (Ti₆Al₄V) cylinder of 14 mm in diameter and 2 mm in height was provided as the substrate. Then, the ZrO₂ layer was formed on the Ti₆Al₄V substrate by the atomic layer deposition as in Example 1, except that the atomic layer deposition method was repeated for more than 200 times to provide ZrO₂ with a thickness of 20 nm. Thereby, a ZrO₂ ceramic layer having a thickness of 20 nm was formed on the Ti₆Al₄V substrate.

### Example 4

In Example 4 the same method as in Example 3 was performed to form the ZrO₂ layer on the Ti₆Al₄V substrate, except that the atomic layer deposition cycle was repeated for more than 1000 times to provide ZrO₂ with a thickness of 100 nm. Thereby, a ZrO₂ ceramic layer having a thickness of 100 nm was formed on the Ti₆Al₄V substrate.

### Example 5

In this Example a stainless steel 316L (316LSS) cylinder of 14 mm diameter and 2 mm height was provided as the substrate. Then, the atomic layer deposition was performed in the atomic layer deposition reactor with tetrakis dimethylamino zirconium (TDMAZ; Zr(N(CH₃)₂)₄) and water as the precursors at 150° C., to form a ZrO₂ layer on the 316LSS substrate. The atomic layer deposition comprised the following steps: (1) application of pulse with zirconium dimethyl ammonium; (2) nitrogen purging; (3) application of pulse of water; and (4) nitrogen purging, which were repeated for more than 50 times to provide ZrO₂ with a thickness of 5 nm. Thereby, a ZrO₂ ceramic layer having a thickness of 5 nm was formed on the 316LSS substrate, and the ZrO₂ ceramic layer was a crystalline ZrO₂ ceramic layer film.

### Example 6

In Example 6 the same method as in Example 5 was performed to form the ZrO₂ layer on the 316LSS substrate, except that the atomic layer deposition cycle was repeated for more than 200 times to provide ZrO₂ with a thickness of 20 nm. Thereby, a ZrO₂ ceramic layer having a thickness of 20 nm was formed on the 316LSS substrate.

### Example 7

In Example 7 the same method as in Example 5 was performed to form the ZrO₂ layer on the 316LSS substrate, except that the atomic layer deposition cycle was repeated for more than 1000 times to provide ZrO₂ with a thickness of 100 nm. Thereby, a ZrO₂ ceramic layer having a thickness of 100 nm was formed on the 316LSS substrate.

### Example 8

In this Example a stainless steel 316L (316LSS) cylinder of 14 mm diameter and 2 mm height was provided as the substrate. Then, the atomic layer deposition method was performed in the atomic layer deposition reactor with tetrakis dimethylamino hafnium (TDMAH; Hf(N(CH₃)₂)₄) and water as the precursors at 150° C., to form an HfO₂ layer on the 316LSS substrate. The atomic layer deposition comprised the following steps: (1) application of pulse of tetrakis dimethylamino hafnium; (2) nitrogen purging; (3) application of pulse of water; and (4) nitrogen purging, which were repeated for more than 50 times to provide HfO₂ with a thickness of 5 nm. Thereby, an HfO₂ ceramic layer having a thickness of 5 nm was formed on the 316LSS substrate, and the HfO₂ ceramic layer was a crystalline HfO₂ ceramic film.

### Example 9

In Example 9 the same method as in Example 8 was performed to form the HfO₂ layer on the 316LSS substrate, except that the atomic layer deposition cycle was repeated for more than 200 times to provide HfO₂ with a thickness of 20 nm. Thereby, an HfO₂ ceramic layer having a thickness of 20 nm was formed on the 316LSS substrate.

### Example 10

In Example 10 the same method as in Example 8 was performed to form the HfO₂ layer was formed on the 316LSS substrate, except that the atomic layer deposition cycle was repeated for more than 1000 times to provide HfO₂ with a thickness of 100 nm. Thereby, an HfO₂ ceramic layer having a thickness of 100 nm was formed on the 316LSS substrate.

### Example 11

In this Example a stainless steel 316L (316LSS) cylinder of 14 mm diameter and 2 mm height was provided as the substrate. Then, the atomic layer deposition was performed in the atomic layer deposition reactor with titanium tetraisopropoxide (TTIP; Ti(OCH(CH₃)₂)₄) and water as the precursors at 250° C., to form an TiO₂ layer on the 316LSS substrate. The atomic layer deposition comprised the following steps: (1) application of pulse of titanium tetraisopropoxide; (2) nitrogen purging; (3) application of pulse of water; and (4) nitrogen purging, which were repeated for more than 167 times to provide TiO₂ with a thickness of 5 nm. Thereby, a TiO₂ ceramic layer having a thickness of 5 nm was formed on the 316LSS substrate, and the TiO₂ ceramic layer was a crystalline TiO₂ ceramic layer film.

### Example 12

In Example 12 the same method as in Example 11 was performed to form the TiO₂ layer on the 316LSS substrate, except that the atomic layer deposition cycle was repeated for more than 667 times to provide TiO₂ with a thickness of 20 nm. Thereby, a TiO₂ ceramic layer having a thickness of 20 nm was formed on the 316LSS substrate.

### Example 13

In Example 12 the same method as in Example 11 was performed to form the TiO₂ layer on the 316LSS substrate, except that the atomic layer deposition cycle was repeated for more than 3334 times to provide TiO₂ with a thickness of 100 nm. Thereby, a TiO₂ ceramic layer having a thickness of 100 nm was formed on the 316LSS substrate.

TABLE 1

| | Substrate | Ceramic | Thickness of | |
|---|---|---|---|---|
| | material | layer | ceramic layer | |
| Example 1 | Ti | ZrO₂ | 20 | nm |
| Example 2 | Ti | ZrO₂ | 100 | nm |
| Example 3 | Ti₆Al₄V | ZrO₂ | 20 | nm |
| Example 4 | Ti₆Al₄V | ZrO₂ | 100 | nm |
| Example 5 | 316LSS | ZrO₂ | 5 | nm |
| Example 6 | 316LSS | ZrO₂ | 20 | nm |
| Example 7 | 316LSS | ZrO₂ | 100 | nm |
| Example 8 | 316LSS | HfO₂ | 5 | nm |
| Example 9 | 316LSS | HfO₂ | 20 | nm |
| Example 10 | 316LSS | HfO₂ | 100 | nm |
| Example 11 | 316LSS | TiO₂ | 5 | nm |
| Example 12 | 316LSS | TiO₂ | 20 | nm |
| Example 13 | 316LSS | TiO₂ | 100 | nm |

| | | | |
|---|---|---|---|
| Comparative | Ti | — | — |
| Example 1 | | | |
| Comparative | Ti₆Al₄V | — | — |
| Example 2 | | | |
| Comparative | 316LSS | — | — |
| Example 3 | | | |

### Test Example 1

### Lactate Dehydrogenase Assay (LDH Assay)

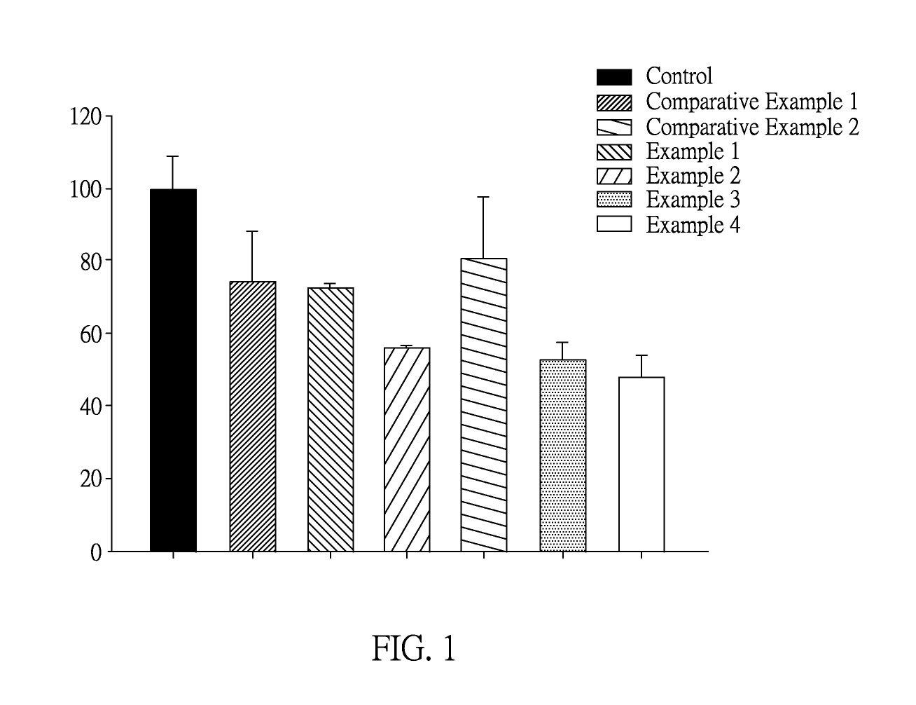

The cylinder samples with the biological ceramics formed thereon prepared in Example 1-4, and the titanium cylinder in Comparative Example 1 and the Ti₆Al₄V cylinder in Comparative Example 2, were immersed in a culture medium for 5 days and the culture medium was replaced twice a day, to remove the substances likely to interfere the experimental results (such as residual HCl, etc.) on the sample surface. Each sample was then placed in a 24-well plate, added with a human osteosarcoma cell (MG-63) having a cell density of 3.3×10⁵ cells/mL, and then incubated for 7 days at 37° C. After that, 50 μL of supernatant was removed to another 96-well plate, added with 50 μL of LDH Cytotoxicity Detection Kit (Takara Bio, Shiga, Japan) and placed in the dark at room temperature for 30 minutes, followed by adding 50 μL of a stop solution (1N HCl) to stop the reaction. Then, the absorbance at 490 nm was measured. The result is shown in FIG. 1, wherein the LDH values of the pure titanium (Ti) or titanium alloy (V) were higher than those of Ti20, Ti100, V20, and V100 with a ZrO₂ film. This results prove that the ZrO₂ film deposited by ALD can reduce the bio-toxicity of the pure titanium (Ti) or titanium alloy (V) substrate, and when the thicker the ZrO₂ film, the higher the cell viability.

### Test Example 2

### X-Ray Photoelectron Spectroscopy (XPS)

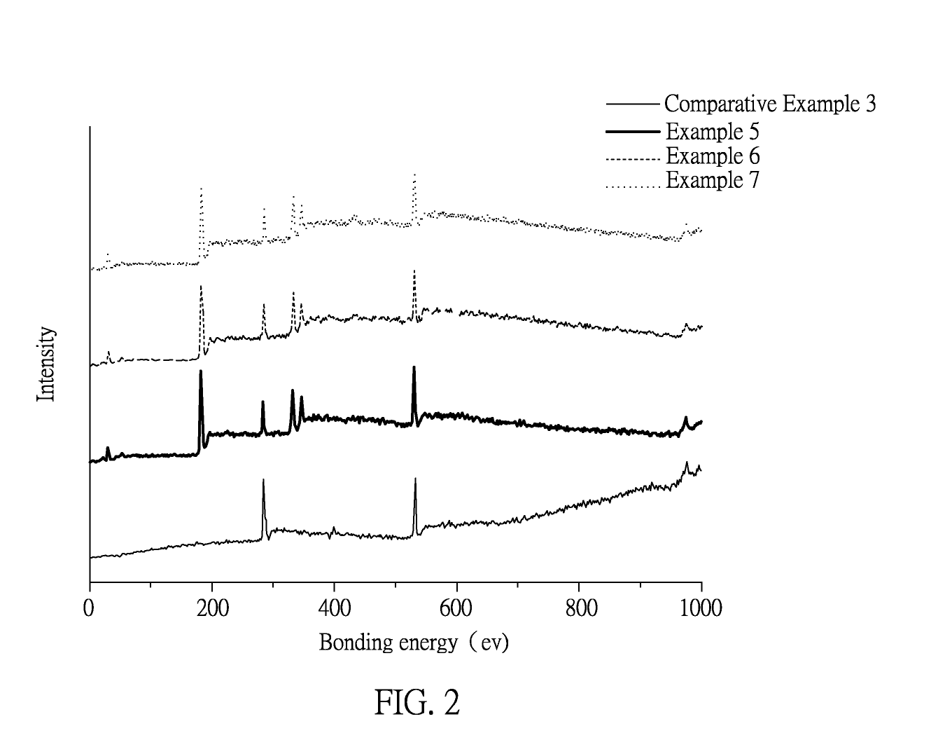

The sample prepared in Example 5 was analyzed using XPS (ULVAC-PHI, Chigasaki, Japan), and the result of the analysis is shown in FIG. 2. FIG. 2 indicates the existence of Zr atoms were on the surfaces of the 316LSS substrates with the ZrO₂ coatings of 5 nm, 20 nm, and 100 nm in thickness, confirming that ZrO₂ was indeed formed on the surface of the 316LSS substrate.

### Test Example 3

### X-Ray Diffraction (XRD)

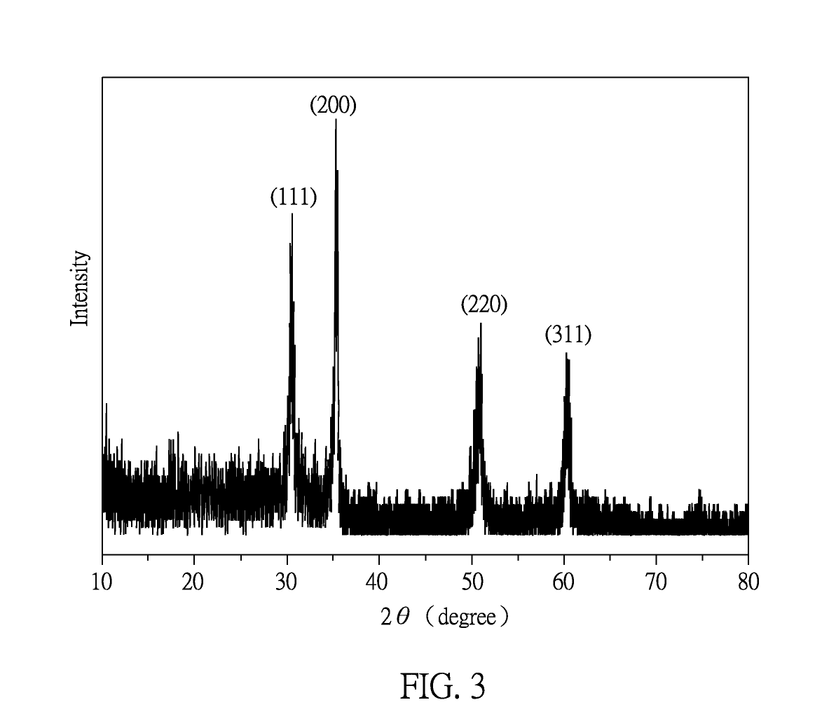

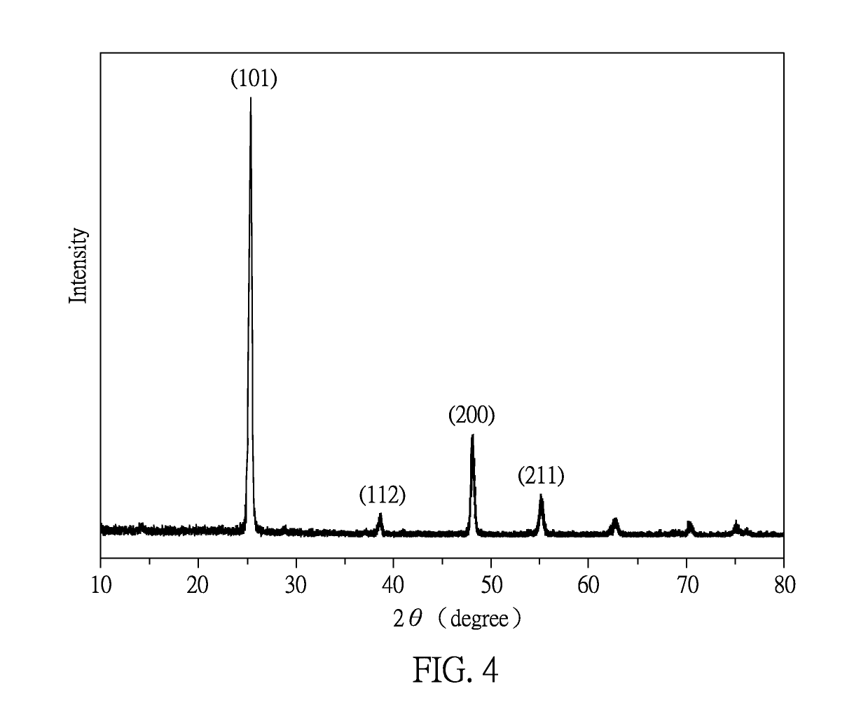

The X-ray diffraction analysis was conducted on the samples prepared in Examples 7 and 13 using an XRD analyzer (TTRAX 3, Rigaku, Japan). According to the analytical results shown in FIG. 3, the ZrO₂ film prepared in accordance with the method in Example 7 had a tetragonal crystalline form. On the other hand, FIG. 4 indicates that the TiO₂ film prepared in accordance with the method in Example 13 had an anatase crystalline form.

### Test Example 4

### Friction and Roughness Analysis

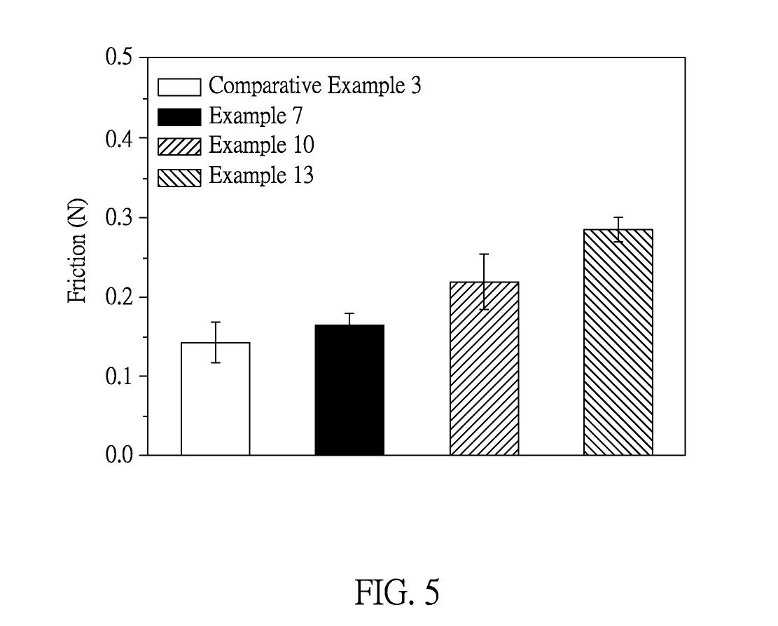

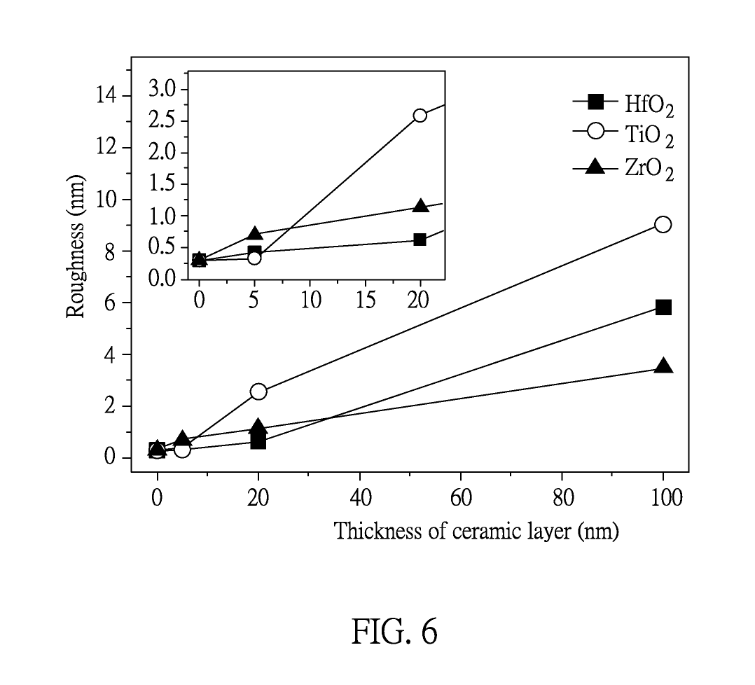

The samples with 100 nm-thick ZrO₂, HfO₂, and TiO₂ on the 346LSS substrate in Examples 7, 10 and 13, were subjected to a friction test, and a 316LSS pristine substrate was used as a control group (Comparative Example 3). According to the result shown in FIG. 5, although the ceramic layers were deposited on the substrate with the same thickness (100 nm), the friction was varied with the types of the ceramic layer, wherein ZrO₂ had the minimal impact on the friction coefficient, HfO₂ second, and TiO₂ had the maximum friction coefficient. In addition, the roughness analysis was performed by an atomic force microscope (MultiMode SPM, Veeco, Santa Barbara, USA). FIG. 6 shows the result of the roughness calculated from five root mean square roughness obtained by randomly scanning five 1 μm×1 μm areas on the surface of each specimen. It can be observed from FIG. 6 that the roughness increased with the thickness of the ceramic layer, and at the deposition thickness of 100 nm, the deposited TiO₂ had the highest roughness, HfO₂ second, and ZrO₂ had the minimum roughness. The friction and roughness analysis indicates that ZrO₂ had a smaller impact on the surface morphology and surface friction coefficient of 316LSS when deposited.

To sum up the results in the above Examples and Test Examples, the present invention provides a surface treatment method for an implant, wherein the thicker the ceramic layer deposited on the surface of the implant, the better the biocompatibility. However, the increase in thickness of the ceramic layer deposited on the implant also brings increased surface roughness and friction. According to the results of the Test Examples, when the ceramic layer deposited on the surface of the implant was ZrO₂, the ZrO₂ had a smaller impact on the roughness and friction coefficient of the implant, and therefore, not only the surface morphology of the implant can be kept more intact during the implanting, but also the biocompatibility of the implant can be improved. In addition, in the surface treatment method for an implant provided by the present invention, the ceramic layer having a crystalline structure may be formed on the surface of the implant by controlling the parameters such as process temperature. As opposed to the amorphous ceramic layer, the crystalline ceramic layer is more difficult to dissolve in water, and thus less likely to dissolve in the body fluid to cause exposure of the implant and deteriorate the biocompatibility, thus increasing the reliability of the implant which needs a long-term contact with the body fluid, such as a dental implant or a cardiovascular stent.

Although the present invention has been explained in relation to its preferred embodiment, it is to be understood that many other possible modifications and variations can be made without departing from the spirit and scope of the invention as hereinafter claimed.

What is claimed is:

1. A surface treatment method for an implant, comprising:
providing an implant; and
forming a ceramic layer on a surface of the implant by atomic layer deposition, wherein the ceramic layer has a thickness of 5-150 nm; a root mean square roughness increase in a range of 20 nm or less; and a friction coefficient of 0.1-0.5.

2. The surface treatment method of claim 1, wherein the ceramic layer has a thickness ranging from 20 to 120 nm.

3. The surface treatment method of claim 1, wherein the ceramic layer has a root mean square roughness increase in a range of 10 nm or less.

4. The surface treatment method of claim 1, wherein the ceramic layer has a friction coefficient ranging from 0.1 to 0.35.

5. The surface treatment method of claim 1, wherein the ceramic layer is formed by repeating the atomic layer deposition for 50-5000 times.

6. The surface treatment method of claim 1, wherein the ceramic layer is made of a metal oxide.

7. The surface treatment method of claim 1, wherein the metal oxide is selected form the group consisting of Al₂O₃, ZnO, TiO₂, ZrO₂, HfO₂ and a mixture thereof.

8. The surface treatment method of claim 1, wherein the atomic layer deposition is performed at a temperature of 150-250° C.

9. The surface treatment method of claim 1, wherein the ceramic layer is in a crystalline structure.

10. The surface treatment method of claim 1, wherein the implant is a dental implant, an orthopedic implant or a cardiovascular stent.

11. A surface-treated implant, comprising:
an implant; and
a ceramic layer formed on a surface of the implant by atomic layer deposition, wherein the ceramic layer has a thickness of 5-150 nm; a root mean square roughness increase in a range of 15 nm or less; and a friction coefficient of 0.1-0.5.

12. The surface-treated implant of claim 11, wherein the ceramic layer has a thickness ranging from 20 to 120 nm.

13. The surface-treated implant of claim 11, wherein the ceramic layer has a root mean square roughness increase in a range of 10 nm or less.

14. The surface-treated implant of claim 11, wherein the ceramic layer has a friction coefficient ranging from 0.1 to 0.35.

15. The surface-treated implant of claim 11, wherein the ceramic layer is made of a metal oxide.

16. The surface-treated implant of claim 11, wherein the metal oxide is selected form the group consisting of Al₂O₃, ZnO, TiO₂, ZrO₂, HfO₂ and a mixture thereof.

17. The surface-treated implant of claim 11, wherein the ceramic layer is in a crystalline structure.

18. The surface-treated implant of claim 11, wherein the implant is a dental implant, an orthopedic implant or a cardiovascular stent.
